(19) United States
(12) Patent Application Publication
(10) Pub. No.: US 2006/0111361 A1
(43) Pub. Date: May 25, 2006

(54) Method of reversing left ventricle remodeling

(76) Inventors: Brent Blackburn, Los Altos, CA (US); Hani Sabbah, Waterford, MI (US)

Correspondence Address: J. Elin Hartrum;CV Therapeutics, Inc., 3172 Porter Drive, Palo Alto, CA, 94304, US

(21) Appl. No.: 11/271,168

(22) Filed: Nov. 9, 2005

Related U.S. Application Data
(60) Provisional application No. 60/626,154, filed on Nov. 9, 2004.

(51) Int. Cl. A61K 31/495; A61K 31/47; A61K 31/4184; A61K 31/403; A61K 31/401; A61K 31/138

(52) U.S. Cl. 514/252.12; 514/411; 514/381; 514/310; 514/423; 514/651

(57) ABSTRACT

The present invention relates to method of reversing left ventricle remodeling by combined administration of therapeutically effective amounts ranolazine and at least one co-remodeling agent, which may be an ACE inhibitor, an ARB, or a beta-blocker. The method finds utility in the treatment of heart failure. This invention also relates to pharmaceutical formulations that are suitable for such combined administration.

### CROSS REFERENCE TO RELATED APPLICATIONS

This application claims priority to U.S. Provisional Patent Application Ser. No. 60/626,154, filed Nov. 9, 2004, the complete disclosure of which is hereby incorporated by reference.

### FIELD OF THE INVENTION

The present invention relates to method of reversing left ventricle remodeling by combined administration of therapeutically effective amounts of ranolazine and at least one co-remodeling agent, which may be an ACE inhibitor, an angiotensin II receptor blocker (ARB), or a beta-blocker. The method finds utility in the treatment of heart failure. This invention also relates to pharmaceutical formulations that are suitable for such combined administration.

### BACKGROUND

Heart failure is a major cause of death and disability in industrialized society. It is not a disease in itself, but a condition in which the heart is unable to pump an adequate supply of blood to meet the oxygen requirements of the body's tissues and organs. As a consequence, fluid often accumulates in the heart and other organs, such as the lungs, and spreads into the surrounding tissues resulting in congestive heart failure (CHF). CHF is often a symptom of cardiovascular problems such as coronary artery disease, myocardial infarction, cardiomyopathy, heart valve abnormalities, and the like.

A significant element of heart failure is the accompanying remodeling of the left ventricle. As the heart muscle fails and loses its ability to pump an adequate supply of blood, the heart, and more specifically the left ventricle (LV), enlarges in an effort to compensate. The extent of this remodeling or enlargement has been correlated with increased mortality rates in heart failure patents and specifically in patents with CHF.

Certain beta-blockers and angiotensin converting enzyme or “ACE” inhibitors have been shown to slow and even reverse the progression of LV remodeling. Both of these agents, however, have undesirable side effects, which limit the dosage amount. Also, there is considerable variability between the ability of different beta-blockers to induce reverse remodeling. There is, therefore, a need to provide a method for increasing reverse LV remodeling. It has now been discovered that administration of Ranolazine and a co-remodeling agent synergistically enhances the reversal of unfavorable left ventricle remodeling.

### SUMMARY OF THE INVENTION

In one embodiment of the invention, a method for reversing unfavorable left ventricle remodeling is provided. The method comprises coadministration of a therapeutically effective amount of ranolazine and a therapeutically effective amount of at least one co-remodeling agent to a mammal in need thereof. The co-remodeling agent may be an ACE inhibitor, an ARB, or a beta-blocker. The method is suitable for use in the treatment of congestive heart failure (CHF) and/or chronic heart failure. Ranolazine and the co-remodeling agent may be administered in separate dosage forms or may be administered in a single dosage form.

In another embodiment of the invention, pharmaceutical formulations are provided comprising a therapeutically effective amount of ranolazine, a therapeutically effective amount at least one co-remodeling agent, and at least one pharmaceutically acceptable carrier.

In yet another embodiment of the invention, a method for treating heart failure in a mammal is provided. The method comprises coadministration of a therapeutically effective amount of ranolazine and a therapeutically effective amount of at least one co-remodeling agent to a mammal in need thereof. The method is suitable for use in the treatment of congestive heart failure (CHF) and/or chronic heart failure. Ranolazine and the co-remodeling agent may be administered in separate dosage forms or may be administered in a single dosage form.

### SUMMARY OF THE FIGURES

FIG. 1 graphically depicts the results of the comparative study of ranolazine, ranolazine and enalapril, and ranolazine and metoprolol tartrate with respect to end-diastolic volume. Historic data on enalapril and metoprolol tartrate is also presented.

FIG. 2 graphically depicts the results of the comparative study of ranolazine, ranolazine and enalapril, and ranolazine and metoprolol tartrate with respect to end-systolic volume. Historic data on enalapril and metoprolol tartrate is also presented.

### DETAILED DISCRIPTION OF THE INVENTION

### Definitions and General Parameters

As used in the present specification, the following words and phrases are generally intended to have the meanings as set forth below, except to the extent that the context in which they are used indicates otherwise.

“Optional” or “optionally” means that the subsequently described event or circumstance may or may not occur, and that the description includes instances where said event or circumstance occurs and instances in which it does not.

The term “ACE inhibitor” refers to an agent that is capable of inhibiting angiotensin converting enzyme, thereby reducing the conversion of angiotensin I to angiotensin II. As a complementary action, ACE inhibitors also reduce the degradation of bradykinin. Suitable ACE inhibitors include, but are not limited to, benazepril, captopril, cilazapril, enalapril, fosinopril, imidapril, lisinopril, perindopril, quinapril, ramipril, temocapril, and trandolapril.

The term “ARB” refers to an agent that is an angiotensin II receptor blocker and are also referred to as angiotensin antagonists. Like ACE inhibitors, ARBs reduce angiotensin II but do it at the cell wall instead of in the blood stream inside the lungs like ACE inhibitors do, thereby acting in a more systemic fashion. Suitable ARBs include, but are not limited to, candesartan, cilexetil, eprosartan, irbesartan, losartan, olmesartan, medoxomil, telmisartan, valsartan, zolasartin, and tasosartan.

The term “beta-blocker” refers to an agent that binds to a beta-adrenergic receptor and inhibits the effects of beta-adrenergic stimulation. Beta-blockers increase AV nodal conduction. In addition, Beta-blockers decrease heart rate by blocking the effect of norepinephrine on the post synaptic nerve terminal that controls heart rate. Beta blockers also decrease intracellular Ca++ overload, which inhibits after-depolarization mediated automaticity. Examples of beta-blockers include, but are not limited to, acebutolol, atenolol, betaxolol, bisoprolol, carteolol, labetalol, metoprolol, nadolol, oxprenolol, penbutolol, pindolol, propranolol, sotalol, timolol, esmolol, sotalol, carvedilol, medroxalol, bucindolol, levobunolol, metipranolol, celiprolol, and propafenone.

“Parenteral administration” is the systemic delivery of the therapeutic agent via injection to the patient.

The term “therapeutically effective amount” refers to that amount of a compound of Formula I that is sufficient to effect treatment, as defined below, when administered to a mammal in need of such treatment. The therapeutically effective amount will vary depending upon the specific activity of the therapeutic agent being used, the severity of the patient's disease state, and the age, physical condition, existence of other disease states, and nutritional status of the patient. Additionally, other medication the patient may be receiving will effect the determination of the therapeutically effective amount of the therapeutic agent to administer.

The term “treatment” or “treating” means any treatment of a disease in a mammal, including:
(i) preventing the disease, that is, causing the clinical symptoms of the disease not to develop;
(ii) inhibiting the disease, that is, arresting the development of clinical symptoms; and/or
(iii) relieving the disease, that is, causing the regression of clinical symptoms.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “unfavorable left ventricular remodeling” refers to alterations in chamber size, wall thickness, and other dimensional changes of the left ventricle and to any other changes to the left ventricle which occur in response to myocardial damage that may be evidenced by decreased diastolic and/or systolic performance.

### THE METHOD OF THE INVENTION

The present invention relates to methods of reversing unfavorable left ventricle remodeling. The method comprises co-administration of a therapeutically effective amount of ranolazine and a therapeutically effective amount of at least one co-remodeling agent to a mammal in need thereof. The co-remodeling agent may be an ACE inhibitor, an ARB, or a beta-blocker. The method is suitable for use in the treatment of congestive heart failure (CHF) and/or chronic heart failure.

Ranolazine and the co-remodeling agent may be administered in separate dosage forms or may be administered in a single dosage form. If administered as separate dosage forms, the separate components can be administered in any order and may be taken simultaneously or staggered.

Ranolazine (±)-N-(2,6-dimethylphenyl)-4-[2-hydroxy-3-(2-methoxyphenoxy)-propyl]-1-piperazineacetamide is an antiischemic agent that is currently undergoing clinical trials for the treatment of angina. The compound itself is disclosed in U.S. Pat. No. 4,567,264, the specification of which is incorporated herein by reference. Sustained release formulations of ranolazine are preferred and are disclosed in U.S. Pat. Nos. 6,503,911, 6,369,062, and 6,617,328.

The ability of ranolazine to provide a benefit in the treatment of heart failure has been previously disclosed in U.S. Pat. Nos. 6,528,511 and 6,528,511 and in Sabbah et al. (2002) J. Card. Fail., 8(6):416-22. The use of ranolazine in the treatment of heart failure in these references is supported by the compound's ability to improve LV function. Prior to the present invention, however, the synergistic ability of the compound to induce reverse LV remodeling when administered with a co-remodeling agent was not known.

Ranolazine and the co-administered agent may be given to the patient in either single or multiple doses by any of the accepted modes of administration of agents having similar utilities, for example as described in those patents and patent applications incorporated by reference, including buccal, by intra-arterial injection, intravenously, intraperitoneally, parenterally, intramuscularly, subcutaneously, orally, or via an impregnated or coated device such as a stent, for example, or an artery-inserted cylindrical polymer.

One mode for administration is parental, particularly by injection. The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the component in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of the components. Administration may be via capsule or enteric coated tablets, or the like. In making the pharmaceutical compositions that include ranolazine and at least one co-administered agent, the active ingredients are usually diluted by an excipient and/or enclosed within such a carrier that can be in the form of a capsule, sachet, paper or other container. When the excipient serves as a diluent, in can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compounds, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions of the invention can be formulated so as to provide quick, sustained or delayed release of the active ingredient after administration to the patient by employing procedures known in the art. As discussed above, given the reduced bioavailabity of ranolazine, sustained release formulations are generally preferred. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902514; and 5,616,345.

The compositions are preferably formulated in a unit dosage form. The term “unit dosage forms” refers to physically discrete units suitable as unitary dosages for human subjects and other mammals, each unit containing a predetermined quantity of the active materials calculated to produce the desired therapeutic effect, in association with a suitable pharmaceutical excipient (e.g., a tablet, capsule, ampoule). The active agents of the invention are effective over a wide dosage range and are generally administered in a pharmaceutically effective amount. It will be understood, however, that the amount of each active agent actually administered will be determined by a physician, in the light of the relevant circumstances, including the condition to be treated, the chosen route of administration, the actual compounds administered and their relative activity, the age, weight, and response of the individual patient, the severity of the patient's symptoms, and the like.

For preparing solid compositions such as tablets, the principal active ingredients are mixed with a pharmaceutical excipient to form a solid preformulation composition containing a homogeneous mixture of a compound of the present invention. When referring to these preformulation compositions as homogeneous, it is meant that the active ingredients are dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage element, the latter being in the form of an envelope over the former. Ranolazine and the co-administered agent(s) can be separated by an enteric layer that serves to resist disintegration in the stomach and permit the inner element to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

The beta blockers, ACE inhibitors, and ARBs of this invention are well known in the art, and are commercially available. Ranolazine may be prepared by conventional methods such as in the manner disclosed in U.S. Pat. No. 4,567,264, the entire disclosure of which is hereby incorporated by reference.

### Example 1

The following example examines the effects of ranolazine alone and in combination with an angiotensin converting enzyme (ACE) inhibitor and in combination with a beta-blocker on the progression of left ventricular (LV) dysfunction and LV chamber remodeling in dogs with chronic heart failure produced by multiple sequential intracoronary microembolizations.

Animal Preparation

Chronic LV dysfunction and failure in dogs was produced by multiple sequential intracoronary embolizations with polystyrene Latex microspheres (77-109 μm in diameter) as previously described by Sabbah et al. (1991) Am. J. Physiol. 260:H1379-H1384. Coronary microembolizations were performed during cardiac catheterization under general anesthesia and sterile conditions. Anesthesia was induced using a combination of intravenous injections of hydromorphone (0.22 mg/kg), diazepam (0.2-0.6 mg/kg) and sodium pentobarbital 50-100 mg to effect. Plane of anesthesia was maintained throughout the study using 1% to 2% isoflurane. Left and right heart catheterization was performed via a femoral arteriotomy and venotomy. After each catheterization, the vessels were repaired using 6-0 silk and the skin closed with 4-0 suture. Microembolizations were discontinued when LV ejection fraction, determined angiographically, was between 30% and 40%. A period of 2 weeks was allowed after the last embolization to ensure that infarctions produced by the last microembolizations have completely healed and heart failure was established. The study protocol was then performed.

Study Animals

Healthy, conditioned, purpose-bred mongrel dogs weighing between 19 and 25 kg.

Study Protocol

A randomized, blinded, placebo controlled study design was used. A total of 28 dogs underwent multiple sequential intracoronary microembolizations as described above to produce chronic heart failure. Two weeks after the last embolization, dogs were randomized into 4 study groups (treatment arms). Dogs were randomized to 3 months oral therapy with ranolazine alone (375 mg, bid, n=7), ranolazine (375 mg bid) in combination with metoprolol tartrate (25 mg bid, n=7), ranolazine (375 mg bid) in combination with enalapril (10 mg bid, n=7), or placebo (ranolazine vehicle bid, n=7). Hemodynamic, angiographic, echocardiographic, Doppler and neurohumoral measurements were made prior to randomization (2 weeks after the last embolization) and after completion of therapy (3 months after initiating therapy). After completing the final hemodynamic and angiographic study, dogs were euthanized and the hearts removed and tissue prepared and saved for future histological and biochemical evaluations. The study primary and secondary end-points were as follows:

Primary Endpoints

Prevention or attenuation of progressive LV dysfunction based on an assessment of LV ejection fraction determined angiographically.

Prevention or attenuation of progressive LV remodeling based on measurements of LV end-diastolic volume and LV end-systolic volume determined angiographically.

Secondary Endpoints

Prevention or attenuation of progressive LV diastolic dysfunction based on assessments of 1) LV peak -dP/dt, 2) LV time constant of early relaxation (Tau), 3) mitral valve velocity PE/PA, and 4) LV end-diastolic circumferential wall stress.

Extent of attenuation of cardiomyocyte hypertrophy, volume fraction of replacement fibrosis, volume fraction of interstitial fibrosis, capillary density and oxygen diffusion distance.

Changes in circulating levels of plasma neurohormones (plasma norepinephrine (PNE), plasma renin activity (PRA) and plasma atrial natriuretic factor (ANF) as well as changes of transmyocardial plasma norepinephrine (arterial to coronary sinus difference).

Hemodynamic Measurements

All hemodynamic measurements were made during left and right heart catheterizations in anesthetized dogs. Baseline measurements, prior to any microembolizations, were be made to ensure that all hemodynamic parameters are within normal limits. Abnormal dogs were excluded from the study. The following parameters were measured in all dogs at all three study time periods: heart rate, mean aortic pressure, peak rate of change of LV pressure during isovolumic contraction (peak +dP/dt) and relaxation (peak −dP/dt), and LV end-diastolic pressure.

Ventriculographic Measurements

Left ventriculograms were performed during cardiac catheterization after completion of the hemodynamic measurements. Ventriculograms were performed with the dog placed on its right side and were recorded on 35 mm cine at 30 frames per second during a power injection of 20 ml of contrast material (RENO-M-60, Squibb Diagnostics). Correction for image magnification was made using a radiopaque grid placed at the level of the LV. LV end-systolic and end-diastolic volumes were calculated from angiographic silhouettes using the area length method (4). Premature beats and postextrasystolic beats were excluded from any analysis. LV ejection fraction was calculated as the ratio of the difference of end-diastolic and end-systolic volumes to end-diastolic volume times 100. Stroke volume was calculated as the difference between LV end-diastolic and end-systolic volumes. Cardiac output was calculated as the stroke volume times heart rate and cardiac index as the cardiac output divided by body surface area.

Echocardiographic and Doppler Measurements

Echocardiographic and Doppler studies were performed in all dogs at all specified study time points using a 77030A ultrasound system (Hewlett-Packard) with a 3.5 MHZ transducer. All echocardiographic measurements were made with the dog placed in the right lateral decubitus position and recorded on a Panasonic 6300 VHS recorder for subsequent off-line analysis. Transverse 2-dimensional echocardiograms were obtained at the level of the LV papillary muscle and were used to calculate LV fractional area of shortening. The latter was calculated as the end-diastolic LV cavity area minus the end-systolic cavity area divided by the end-diastolic cavity area times 100. Two chamber view 2-dimensional echocardiograms were also obtained to ascertain LV major and minor semiaxes to be used for calculation of LV end-diastolic circumferential wall stress. Wall stress was calculated as follows: Stress=Pb/h(1−h/2b)(1−hb/2a2), where P is LV end-diastolic pressure, a is LV major semiaxis, b is LV minor semiaxis, and h is LV wall thickness.

Mitral inflow velocity was measured by pulsed-wave Doppler echocardiography. The velocity waveforms were used to calculate peak mitral flow velocity in early diastole (PE), peak mitral inflow velocity during LA contraction (PA), the ratio of PE to PA and early mitral inflow deceleration time. The presence or absence of functional mitral regurgitation (MR) was determined with Doppler color flow mapping (Hewlett-Packard model 77020A Ultrasound System) using both an apical two-chamber and an apical four-chamber views. When present, the severity of functional MR was quantified based on the ratio of the regurgitant jet area to the area of the left atrium times 100. The ratios calculated from both views were then averaged to obtain single representative measure of the severity of functional MR.

Blood Neurohumoral and Electrolyte Measurements

Evaluation of plasma concentrations of several neurohormones were made to complement the hemodynamic assessments. Measurements were made at each of the study time periods described for hemodynamic and angiographic assessments. Transmyocardial PNE concentration was estimated by obtaining blood samples from the ascending aorta and coronary sinus during cardiac catheterization. Transmyocardial PNE was calculated as the difference between the two samples. Venous blood samples were obtained in duplicate from conscious dogs prior to cardiac catheterizations for measurement of plasma concentration of norepinephrine, plasma renin activity and plasma atrial natriuretic factor using radioimmunoassay. In addition, blood samples were obtained at the same time intervals for determination of serum electrolytes (Na+, K+, creatinine and BUN).

Histomorphometric Evaluations

On the day of sacrifice, after completion of all hemodynamic and angiographic studies, the dog's chest was opened through a left thoracotomy, the pericardium was opened and the heart rapidly removed and placed in ice-cold, Tris buffer (pH 7.4). Three 2 mm thick transverse slices were obtained from the LV; one slice from the basal third, one from the middle third and one from the apical third and placed in 10% formalin. Transmural blocks were also obtained and rapidly frozen in isopentane cooled to −160° C. by liquid nitrogen and stored at −70° C. until needed. LV tissue samples were also obtained and stored in gluteraldehyde for future scanning and transmission electron microscopic studies.

From each heart, 3 transverse slices one from the basal third, middle third and apical third of the LV, each approximately 3 mm thick, were obtained. For comparison, tissue samples from 7 normal dogs were obtained and prepared in an identical manner. From each transverse slice, transmural tissue blocks were obtained and embedded in paraffin blocks. From each block, 6 μm thick sections were prepared and stained with Gomori trichrome to identify fibrous tissue. The volume fraction of replacement fibrosis namely, the proportion of scar tissue to viable tissue in all three transverse LV slices, was calculated as the percent total surface area occupied by fibrous tissue using computer-based video densitometry (MOCHA, Jandel Scientific, Corte Madera, Calif.). LV free wall tissue blocks were obtained from a second mid-ventricular transverse slice, were mounted on cork using Tissue-Tek embedding medium (Sakura, Torrance, Calif.) and rapidly frozen in isopentane pre-cooled in liquid nitrogen and stored at −70° C. until used. Cryostat sections, approximately 8 μm thick, were prepared from each block and stained with fluorescein-labeled peanut agglutinin (Vector Laboratories Inc., Burlingame, Calif.) after pretreatment with 3.3 U/ml neuroaminidase type V (Sigma Chemical Co., St. Louis. Mo.) to delineate the myocyte border and the interstitial space including capillaries (5). Sections were double stained with rhodamine-labeled Griffonia simplicifolia lectin I (GSL-I) to identify capillaries. Ten radially oriented microscopic fields (magnification X100, objective X40, and ocular 2.5) were selected at random from each section for analysis. Fields containing scar tissue (infarcts) were excluded. An average myocyte cross-sectional area was calculated for each dog using computer-assisted planimetry. The total surface area occupied by interstitial space and the total surface are occupied by capillaries were measured from each randomly selected field using computer-based video densitometry (MOCHA, Jandel Scientific, Corte Madera, Calif.). The volume fraction of interstitial collagen was calculated as the percent total surface area occupied by interstitial space minus the percent total area occupied by capillaries (5). Capillary density was calculated as the number of capillaries per mm2. Oxygen diffusion distance was calculated as half the distance between two adjoining capillaries. For comparison, identical measurements were made using LV tissue obtained from 7 normal dogs.

Statistical Analysis

To ensure that all study measures were similar at baseline, comparisons were made between all 4 study groups before any embolizations and at the time of randomization before initiation of therapy. For these comparisons, a one way analysis of variance (ANOVA) was used with a set at 0.05. If significance was attained, then group wise comparisons were made using the Student-Newman-Kuels test with significance set at p≦0.05. Within group comparisons between pre-treatment and post-treatment measures were made using a Students paired t-test with p<0.05 considered significant. To assess treatment effect, the change (Δ) in each measure from pre-treatment to post-treatment was calculated for each of the 4 study arms. To determine whether significant differences in Δ were present between groups, ANOVA was used with a set at 0.05. If significance was attained, then group wise comparisons were made using the Student-Newman-Kuels test with significance set at p≦0.05. All data are reported as the mean ±SEM.

Baseline Measures

Baseline hemodynamic, ventriculographic, echocardiographic, Doppler and plasma neurohormones and electrolytes measures obtained prior to any microembolizations are shown in tables 1 through 4. There were no significant differences among the 4 study groups in any of the baseline measures.

Pre-Treatment Measures

Hemodynamic, ventriculographic, echocardiographic, Doppler and plasma neurohormones and electrolytes measures obtained at the time or randomization are shown in tables 5 through 8. There were no significant differences among the 4 study groups in any of the pre-treatment measures.

Intra-Group Comparisons—Placebo (Tables 5-8)

In dogs randomized to placebo, there were no differences in heart rate, mean aortic pressure and LV end-diastolic pressure between pre-treatment and post-treatment. However, both LV peak +dP/dt and −dP/dt decreased significantly. In this study group, LV end-diastolic volume and end-systolic volume increased significantly at the end of 3 months of treatment while LV ejection fraction and stroke volume decreased significantly. Cardiac output and cardiac index tended to also decrease but the reduction did not reach statistical difference. Echocardiographic and Doppler results showed significant reduction in LV fractional area of shortening, mitral inflow PE/PA ratio and deceleration time with significant increases in the severity of function mitral regurgitation and LV end-diastolic circumferential wall stress. There were no significant differences in plasma neurohormones and electrolytes.

Intra-Group Comparisons—Ranolazine Alone (Tables 5-8)

In dogs randomized to monotherapy with ranolazine, there were no differences in heart rate, mean aortic pressure, LV peak +dP/dt and peak −dP/dt but LV end-diastolic pressure decreased significantly. In this study group, LV end-diastolic volume and end-systolic volume remained unchanged at the end of 3 months of treatment while LV ejection fraction, stroke volume and cardiac index increased significantly. Cardiac output tended to also increase but the increase did not reach statistical difference. Echocardiographic and Doppler results showed significant no change in LV fractional area of shortening, mitral inflow PE/PA ratio, deceleration time and severity of function mitral regurgitation. LV end-diastolic circumferential wall stress, however, decreased significantly. There were no significant differences in plasma neurohormones and electrolytes.

Intra-Group Comparisons—Ranolazine+Enalapril (Tables 5-8)

In dogs randomized to combination therapy with ranolazine and enalapril, there were no differences in heart rate, mean aortic pressure, LV peak +dP/dt and peak −dP/dt but LV end-diastolic pressure decreased significantly. In this study group, LV end-diastolic volume remained unchanged and end-systolic volume decreased significantly at the end of 3 months of treatment while LV ejection fraction, stroke volume and cardiac index increased significantly. Cardiac output tended to also increase but the increase did not reach statistical difference. Echocardiographic and Doppler results showed significant increase in LV fractional area of shortening and deceleration time. The PE/PA ratio tended to increase but did not reach statistical difference. The severity of functional mitral regurgitation and LV end-diastolic circumferential wall stress decreased significantly. There were no significant differences in plasma neurohormones and electrolytes.

Intra-Group Comparisons—Ranolazine+Metprolol (Tables 5-8)

In dogs randomized to combination therapy with ranolazine and metoprolol, there were no differences in heart rate, mean aortic pressure, LV peak +dP/dt and peak −dP/dt but LV end-diastolic pressure decreased significantly. In this study group, LV end-diastolic volume and end-systolic volume decreased significantly at the end of 3 months of treatment while LV ejection fraction, stroke volume and cardiac index increased significantly. Cardiac output tended to also increase but the increase did not reach statistical difference. Echocardiographic and Doppler results showed significant increase in LV fractional area of shortening, PE/PA ratio and deceleration time. The severity of functional mitral regurgitation and LV end-diastolic circumferential wall stress decreased significantly. There were no significant differences in plasma neurohormones and electrolytes.

Treatment Effect—Inter-Group Comparisons (Tables 9-12)

Treatment effect data are shown in tables 9 through 12 and individual dog data are shown in Appendix 1. Treatment effect analysis showed no differences among the 4 study groups with respect to heart rate and mean aortic pressure. Compared to placebo, LV end-diastolic pressure, peak LV +dP/dt and peak −dP/dt increased significantly in dogs treated with ranolazine alone and with ranolazine combined with either enalapril or metoprolol. LV end-diastolic, end-systolic volume, ejection fraction, stroke volume and cardiac index all improved significantly in all 3 treatment arms compared to placebo. Cardiac output tended to also increase in the treatment arms compared to placebo but the increase did not reach statistical difference. The reductions in LV volumes and the increase in LV ejection fraction were significantly greater in dogs randomized to combination therapy compared to dogs randomized to ranolazine alone.

Compared to placebo, ranolazine alone significantly increase LV fractional area of shortening and significantly reduced LV end-diastolic wall stress. PE/PA ratio, severity of MR and deceleration time tended to improve with ranolazine alone compared to placebo but the extent of improvement did not reach statistical difference. Compared to placebo, combination therapies significantly improved LV fractional area of shortening, PE/PA ratio, severity of mitral regurgitation, deceleration time and LV end-diastolic wall stress. There were no significant differences among the 4 study groups with respect to plasma neurohormones and electrolytes.

Histomorphometric Findings

Histomorphometric data are shown in table 13. Compared to normal dogs, dogs treated with placebo showed a significant increase in myocyte cross-sectional area, volume fraction of replacement and interstitial fibrosis and oxygen diffusion distance along with a significant decrease in capillary density. Treatment with ranolazine alone as well as treatment with combination therapy significantly improved all of the above histomorphometric measures compared to placebo. The extent of improvement was significantly greater in dogs treated with combination therapy that those treated with ranolazine alone.

The results of this study performed in dogs with moderate heart failure indicate that monotherapy with ranolazine prevents the progression of heart failure as evidenced by preservation of LV function and attenuation of LV remodeling. When combined with an ACE inhibitor or a beta-blocker, ranolazine markedly improves LV systolic and diastolic function and elicits reversal of global and cellular LV remodeling as evidenced by reduction in LV size and improvement in myocyte hypertrophy, interstitial fibrosis, capillary density and oxygen diffusion distance. The results support the use of ranolazine as adjunct therapy for treatment of chronic heart failure.

TABLE 1
Hemodynamic measures at baseline prior to any microembolizations.

| | Placebo | RAN Alone | RAN + ENA | RAN + MET |
|---|---|---|---|---|
| Heart Rate (beats/min) | 83 ± 3 | 81 ± 3 | 92 ± 5 | 80 ± 6 |
| Mean Aortic Pressure (mmHg) | 81 ± 3 | 75 ± 3 | 74 ± 4 | 75 ± 5 |
| LV End-Diastolic Pressure (mmHg) | 7 ± 1 | 8 ± 1 | 7 ± 1 | 8 ± 1 |
| Peak LV +dP/dt (mmHg/sec) | 1481 ± 51 | 1590 ± 109 | 1321 ± 72 | 1579 ± 91 |
| Peak LV −dP/dt (mmHg/sec) | 2024 ± 121 | 1921 ± 158 | 1779 ± 154 | 2074 ± 150 |

LV = left ventricular;
RAN = ranolazine;
ENA = enalapril;
MET = metoprolol

TABLE 2
Ventriculographic measures at baseline prior to any microembolizations.

| | Placebo | RAN Alone | RAN + ENA | RAN + MET |
|---|---|---|---|---|
| LV End-Diastolic Volume (ml) | 49 ± 2 | 50 ± 2 | 48 ± 2 | 50 ± 2 |
| LV End-Systolic Volume (ml) | 23 ± 1 | 24 ± 1 | 23 ± 1 | 24 ± 1 |
| LV Ejection Fraction (%) | 53 ± 1 | 53 ± 1 | 53 ± 1 | 52 ± 1 |
| Stroke Volume (ml) | 26 ± 1 | 27 ± 1 | 25 ± 1 | 26 ± 1 |
| Cardiac Output (L/min) | 2.18 ± 0.12 | 2.17 ± 0.12 | 2.34 ± 0.22 | 2.11 ± 0.21 |
| Cardiac Index (L/min/m2) | 2.8 ± 0.2 | 2.7 ± 0.1 | 3.1 ± 0.3 | 2.7 ± 0.3 |

LV = left ventricular;
RAN = ranolazine;
ENA = enalapril;
MET = metoprolol

TABLE 3
Echocardiographic and Doppler measures at baseline prior to any
microembolizations.

| | Placebo | RAN Alone | RAN + ENA | RAN + MET |
|---|---|---|---|---|
| LV Fractional Area of Shortening (%) | 47 ± 3 | 47 ± 2 | 45 ± 1 | 45 ± 2 |
| PE/PA Ratio | 3.4 ± 0.2 | 4.7 ± 0.3 | 3.2 ± 0.4 | 3.2 ± 0.3 |
| Severity of MR (%) | 0.5 ± 0.5 | 0.9 ± 0.6 | 0.8 ± 0.5 | 1.1 ± 0.7 |
| Deceleration Time (msec) | 113 ± 5 | 115 ± 9 | 115 ± 8 | 108 ± 4 |
| LV EDWS (g-cm2) | 28 ± 3 | 25 ± 2 | 25 ± 2 | 28 ± 3 |

LV = left ventricular;
RAN = ranolazine;
ENA = enalapril;
MET = metoprolol;
EDWS = end-diastolic circumferential wall stress

TABLE 4
Neurohumoral and electrolyte measures at baseline prior to any
microembolizations.

| | Placebo | RAN Alone | RAN + ENA | RAN + MET |
|---|---|---|---|---|
| Na+ (mmol/L) | 147 ± 1 | 147 ± 1 | 148 ± 1 | 147 ± 1 |
| K+ (mmol/L) | 4.7 ± 0.1 | 4.8 ± 0.2 | 4.7 ± 0.1 | 4.4 ± 0.1 |
| Creatinine (mg/dL) | 0.9 ± 0.0 | 0.8 ± 0.0 | 0.8 ± 0.0 | 0.9 ± 0.0 |
| BUN (mg/dL) | 15 ± 2 | 15 ± 3 | 15 ± 1 | 14 ± 2 |
| Plasma Norepinephrine (pg/ml) | 330 ± 61 | 297 ± 60 | 250 ± 56 | 151 ± 28 |
| Plasma Renin Activity (ng/ml/hr) | 1.32 ± 0.21 | 2.35 ± 0.56 | 1.87 ± 0.57 | 3.13 ± 1.16 |
| Plasma ANF (pg/ml) | 71 ± 11 | 81 ± 19 | 51 ± 8 | 60 ± 11 |
| Transmyocardial PNE (pg/ml) | 0 ± 7 | 16 ± 10 | 20 ± 22 | 20 ± 7 |

LV = left ventricular;
RAN = ranolazine;
ENA = enalapril;
MET = metoprolol;
ANF = atrial natriuretic factor;
PNE = plasma norepinephrine

TABLE 5
Hemodynamic measures at time of randomization (PRE) and after 3 months of therapy (POST).

| | Placebo | | RAN Alone | | RAN + ENA | | RAN + MET | |
|---|---|---|---|---|---|---|---|---|
| | PRE | POST | PRE | POST | PRE | POST | PRE | POST |
| HR | 79 ± 5 | 82 ± 5 | 77 ± 2 | 80 ± 4 | 82 ± 4 | 84 ± 4 | 84 ± 2 | 80 ± 3 |
| mAoP | 77 ± 4 | 72 ± 3 | 72 ± 3 | 81 ± 7 | 74 ± 4 | 76 ± 5 | 73 ± 3 | 72 ± 5 |
| LVEDP | 14 ± 1 | 15 ± 1 | 14 ± 1 | 10 ± 1* | 13 ± 1 | 9 ± 1* | 14 ± 1 | 7 ± 1* |
| +dP/dt | 1287 ± 74 | 1080 ± 203* | 1253 ± 119 | 1416 ± 124 | 1079 ± 77 | 1349 ± 141 | 1151 ± 104 | 1273 ± 103 |
| −dP/dt | 1458 ± 110 | 1188 ± 68* | 1254 ± 74 | 1460 ± 134 | 1161 ± 82 | 1514 ± 205 | 1416 ± 69 | 1710 ± 152 |

RAN = ranolazine;
ENA = enalapril;
MET = metoprolol;
HR = heart rate (beats/min);
mAoP = mean aortic pressure (mmHg);
LVEDP = LV end-diastolic pressure (mmHg);
+dP/dt = peak LV +dP/dt (mmHg/sec);
−dP/dt = peak LV −dP/dt (mmHg/sec);
*= p < 0.05

TABLE 6
Ventriculographic measures at time of randomization (PRE) and after 3 months of therapy (POST).

| | Placebo | | RAN Alone | | RAN + ENA | | RAN + MET | |
|---|---|---|---|---|---|---|---|---|
| | PRE | POST | PRE | POST | PRE | POST | PRE | POST |
| EDV | 60 ± 2 | 69 ± 2* | 63 ± 3 | 65 ± 2 | 59 ± 3 | 59 ± 3 | 61 ± 1 | 59 ± 1* |
| ESV | 38 ± 2 | 50 ± 2* | 41 ± 2 | 41 ± 2 | 39 ± 2 | 35 ± 2* | 40 ± 1 | 35 ± 1* |
| EF | 36 ± 1 | 28 ± 1* | 35 ± 1 | 37 ± 2* | 35 ± 1 | 40 ± 1* | 34 ± 1 | 41 ± 1* |
| SV | 22 ± 1 | 19 ± 1* | 22 ± 1 | 24 ± 1* | 21 ± 1 | 24 ± 1* | 20 ± 1 | 24 ± 1* |
| CO | 1.71 ± .14 | 1.53 ± .08 | 1.67 ± .11 | 1.91 ± .18 | 1.69 ± .08 | 1.98 ± .15 | 1.70 ± .06 | 1.92 ± .07 |
| CI | 2.2 ± 0.2 | 1.9 ± 0.1 | 2.0 ± 0.2 | 2.3 ± 0.2* | 2.2 ± 0.1 | 2.6 ± 0.2* | 2.1 ± 0.1 | 2.4 ± 0.1* |

RAN = ranolazine;
ENA = enalapril;
MET = metoprolol;
EDV = LV end-diastolic volume (ml);
ESV = LV end-systolic volume (ml);
EF = LV ejection fraction (%);
SV = stroke volume (ml);
CO = cardiac output (L/min);
CI = cardiac index (L/min/m²);
*= p < 0.05

TABLE 7
Echocardiographic and Doppler measures at time of randomization (PRE) and after 3 months of
therapy (POST).

| | Placebo | | RAN Alone | | RAN + ENA | | RAN + MET | |
|---|---|---|---|---|---|---|---|---|
| | PRE | POST | PRE | POST | PRE | POST | PRE | POST |
| FAS | 29 ± 2 | 24 ± 1* | 28 ± 1 | 29 ± 2 | 28 ± 2 | 33 ± 2* | 27 ± 2 | 33 ± 2* |
| PE/PA | 2.8 ± 0.2 | 2.2 ± 0.2* | 2.7 ± 0.3 | 2.8 ± 0.3 | 2.4 ± 0.3 | 3.1 ± 0.4 | 2.4 ± 0.2 | 3.1 ± 0.3* |
| MR | 11.6 ± 2.3 | 14.1 ± 2.0* | 11.3 ± 1.9 | 10.1 ± 1.3 | 11.9 ± 2.9 | 6.7 ± 1.9* | 10.6 ± 1.8 | 6.0 ± 1.3* |
| DT | 81 ± 3 | 67 ± 3* | 88 ± 3 | 87 ± 4 | 78 ± 4 | 90 ± 5* | 78 ± 6 | 95 ± 5* |
| EDWS | 61 ± 4 | 67 ± 4 | 56 ± 5 | 42 ± 4* | 51 ± 3 | 38 ± 3* | 53 ± 4 | 33 ± 4* |

RAN = ranolazine;
ENA = enalapril;
MET = metoprolol;
FAS = LV fractional area of shortening (%);
MR = severity of mitral regurgitation (%);
DT = deceleration time (msec);
EDWS = LV end-diastolic circumferential wall stress (gm-cm²);
*= p < 0.05

TABLE 8
Neurohumoral and electrolyte measures at time of randomization (PRE)
and after 3 months of therapy (POST).

| | Placebo | | RAN Alone | | RAN + ENA | | RAN + MET | |
|---|---|---|---|---|---|---|---|---|
| | PRE | POST | PRE | POST | PRE | POST | PRE | POST |
| Na+ | 148 ± 0.4 | 148 ± 0.7 | 147 ± 0.5 | 147 ± 0.8 | 148 ± 1.0 | 147 ± 0.7 | 148 ± 0.9 | 147 ± 0.8 |
| K+ | 4.6 ± 0.3 | 4.5 ± 0.1 | 4.5 ± 0.1 | 4.7 ± 0.1 | 4.5 ± 0.1 | 4.7 ± 0.1 | 4.6 ± 0.2 | 4.6 ± 0.1 |
| Creat | 0.9 ± 0.1 | 0.9 ± 0.0 | 0.9 ± 0.0 | 1.0 ± 0.1 | 0.9 ± 0.0 | 0.9 ± 0.0 | 0.9 ± 0.0 | 0.9 ± 0.0 |
| BUN | 16 ± 2 | 13 ± 1 | 18 ± 2 | 15 ± 2 | 17 ± 1 | 16 ± 2 | 18 ± 3 | 14 ± 1 |
| PNE | 137 ± 22 | 200 ± 43 | 217 ± 25 | 199 ± 43 | 213 ± 40 | 212 ± 45 | 190 ± 40 | 159 ± 31 |
| PRA | 2.02 ± .69 | 2.04 ± .09 | 1.69 ± .71 | 1.57 ± .55 | 1.47 ± .30 | 3.49 ± 1.04 | 1.85 ± .42 | 1.82 ± .56 |
| ANF | 73 ± 8 | 78 ± 3 | 87 ± 15 | 67 ± 11 | 86 ± 16 | 78 ± 3 | 99 ± 13 | 84 ± 12 |
| T-PNE | −7.1 ± 3.6 | −2.9 ± 5.7 | 8.6 ± 7.0 | −2.9 ± 6.1 | 5.7 ± 10.9 | −8.6 ± 5.5 | −2.9 ± 3.6 | −14.3 ± 8.1 |

RAN = ranolazine;
ENA = enalapril;
MET = metoprolol;
Creat = serum creatinine (mg/dL);
BUN = blood urea nitrogen (mg/dL);
PNE = plasma norepinephrine (pg/ml);
PRA = plasma rennin activity (ng/ml/hr);
ANF = atrial natriuretic factor (pg/ml);
T-PNE = transmyocardial norepinephrine

Treatment Effect Tables

TABLE 9
Comparison of the change (Δ) from pre-treatment to post-treatment in
hemodynamic measurements between the 4 study
groups (Treatment Effect)

| | Placebo | RAN Alone | RAN + ENA | RAN + MET |
|---|---|---|---|---|
| Δ HR | 3.1 ± 8.3 | 3.3 ± 4.9 | 2.0 ± 4.5 | −2.9 ± 3.1 |
| Δ mAoP | −5.6 ± 3.2 | 9.1 ± 6.9 | 1.7 ± 7.7 | −1.6 ± 6.6 |
| Δ LVEDP | 1.0 ± 1.0 | −4.4 ± 1.1* | −4.3 ± 0.6* | −6.9 ± 1.1* |
| Δ +dP/dt | −207 ± 61 | 163 ± 81* | 270 ± 121* | 121 ± 66* |
| Δ −dP/dt | −270 ± 102 | 206 ± 109* | 353 ± 167* | 294 ± 135* |

RAN = ranolazine;
ENA = enalapril;
MET = metoprolol;
HR = heart rate (beats/min);
mAoP = mean aortic pressure (mmHg);
LVEDP = LV end-diastolic pressure (mmHg);
+dP/dt = peak LV +dP/dt (mmHg/sec);
−dP/dt = peak LV −dP/dt (mmHg/sec);
*= p < 0.05 vs. Placebo

TABLE 10
Comparison of the change (Δ) from pre-treatment to post-treatment in
ventriculographic measures between the 4 study groups (Treatment Effect)

| | Placebo | RAN Alone | RAN + ENA | RAN + MET |
|---|---|---|---|---|
| Δ EDV | 9 ± 1 | 2 ± 1* | −1 ± 1*† | −2 ± 1*† |
| Δ ESV | 12 ± 1 | 0 ± 1* | −3 ± 1*† | −5 ± 1*† |
| Δ EF | −9 ± 1 | 2 ± 1* | 5 ± 1*† | 8 ± 1*† |
| Δ SV | −3 ± 1 | 2 ± 1* | 3 ± 1* | 4 ± 1* |
| Δ CO | −0.18 ± 0.16 | 0.24 ± .10 | 0.29 ± 0.14 | 0.22 ± 0.09 |
| Δ CI | −0.3 ± 0.2 | 0.3 ± 0.1* | 0.4 ± 0.2* | 0.4 ± 0.1* |

RAN = ranolazine;
ENA = enalapril;
MET = metoprolol;
EDV = LV end-diastolic volume (ml);
ESV = LV end-systolic volume (ml);
EF = LV ejection fraction (%);
SV = stroke volume (ml);
CO = cardiac output (L/min);
CI = cardiac index (L/min/m²);
*= p < 0.05 vs. Placebo;
†= p < 0.05 vs. RAN Alone.

TABLE 11
Comparison of the change (Δ) from pre-treatment to post-treatment in
echocardiographic and Doppler measures between the 4 study
groups (Treatment Effect)

| | Placebo | RAN Alone | RAN + ENA | RAN + MET |
|---|---|---|---|---|
| Δ FAS | −5 ± 2 | 1 ± 1* | 5 ± 2* | 6 ± 2* |
| Δ PE/PA | −0.6 ± 0.2 | 0.0 ± 0.4 | 0.7 ± 0.3* | 0.7 ± 0.2* |
| Δ MR | 2.5 ± 0.9 | −1.2 ± 1.1 | −5.2 ± 2.2* | −4.62 ± 1.4* |
| Δ DT | −14 ± 4 | −1 ± 4 | 12 ± 6* | 17 ± 7* |
| Δ EDWS | 6.7 ± 4 | −14.5 ± 5.2* | −13.3 ± 3.3* | −20.4 ± 3.5* |

RAN = ranolazine;
ENA = enalapril;
MET = metoprolol;
FAS = LV fractional area of shortening (%);
MR = severity of mitral regurgitation (%);
DT = deceleration time (msec);
EDWS = LV end-diastolic circumferential wall stress (gm-cm²);
*= p < 0.05 vs. Placebo.

TABLE 12
Comparison of the change (Δ) from pre-treatment to post-treatment in
neurohumoral and electrolyte measures between the 4 study
groups (Treatment Effect)

| | Placebo | RAN Alone | RAN + ENA | RAN + MET |
|---|---|---|---|---|
| Δ Na+ | 1.1 ± 0.9 | −0.3 ± 1.0 | −1.0 ± 1.2 | −0.5 ± 1.2 |
| Δ K+ | −0.1 ± 0.2 | 0.1 ± 0.1 | 0.2 ± 0.1 | 0.0 ± 0.2 |
| Δ Creat | 0.0 ± 0.0 | 0.0 ± 0.1 | 0.0 ± 0.1 | 0.0 ± 0.0 |
| Δ BUN | −2.9 ± 1.3 | −2.1 ± 1.4 | −1.8 ± 2.2 | −4.2 ± 1.9 |
| Δ PNE | 52 ± 33 | −19 ± 42 | −15 ± 61 | −31 ± 35 |
| Δ PRA | 0.03 ± 0.64 | 0.13 ± 0.92 | 2.02 ± 1.04 | −0.45 ± 0.30 |
| Δ ANF | 7 ± 8 | 20 ± 14 | −7 ± 19 | −15 ± 17 |
| Δ T-PNE | 4 ± 7 | −11 ± 9 | −14 ± 11 | −11 ± 6 |

RAN = ranolazine;
ENA = enalapril;
MET = metoprolol;
Creat = serum creatinine (mg/dL);
BUN = blood urea nitrogen (mg/dL);
PNE = plasma norepinephrine (pg/ml);
PRA = plasma rennin activity (ng/ml/hr);
ANF = atrial natriuretic factor (pg/ml);
T-PNE = transmyocardial norepinephrine.

TABLE 13
Histomorphometric Measurements

| | Normal | Placebo | RAN Alone | RAN + ENA | RAN + MET |
|---|---|---|---|---|---|
| MCSA (μm2) | 409 ± 10 | 772 ± 21* | 685 ± 23*† | 558 ± 11*†‡ | 571 ± 14*†‡ |
| VFRF (%) | 0 ± 0 | 14.6 ± 1.2* | 10.5 ± 2.1*† | 9.4 ± 0.9*† | 9.3 ± 1.1*†‡ |
| VFIF (%) | 3.7 ± 0.1 | 13.0 ± 0.7* | 11.0 ± 0.4*† | 8.8 ± 0.6*†‡ | 7.8 ± 0.7*†‡ |
| CD (capillaries/mm2) | 2607 ± 80 | 1706 ± 28* | 1832 ± 43*† | 2059 ± 82*† | 2049 ± 80*† |
| ODD (μm) | 8.9 ± 0.2 | 11.5 ± 0.3* | 10.9 ± 0.2* | 10.4 ± 0.4* | 10.6 ± 0.5* |

RAN = ranolazine;
ENA = enalapril;
MET = metoprolol;
MCSA = myocyte cross-sectional area;
VFRF = volume fraction of replacement fibrosis;
VFIF = volume fraction of interstitial fibrosis;
CD = capillary density;
ODD = oxygen diffusion distance;
*= p < 0.05 vs. Normal;
†= p < 0.05 vs. Placebo;
‡= p < 0.05 vs. RAN Alone

### Example 2

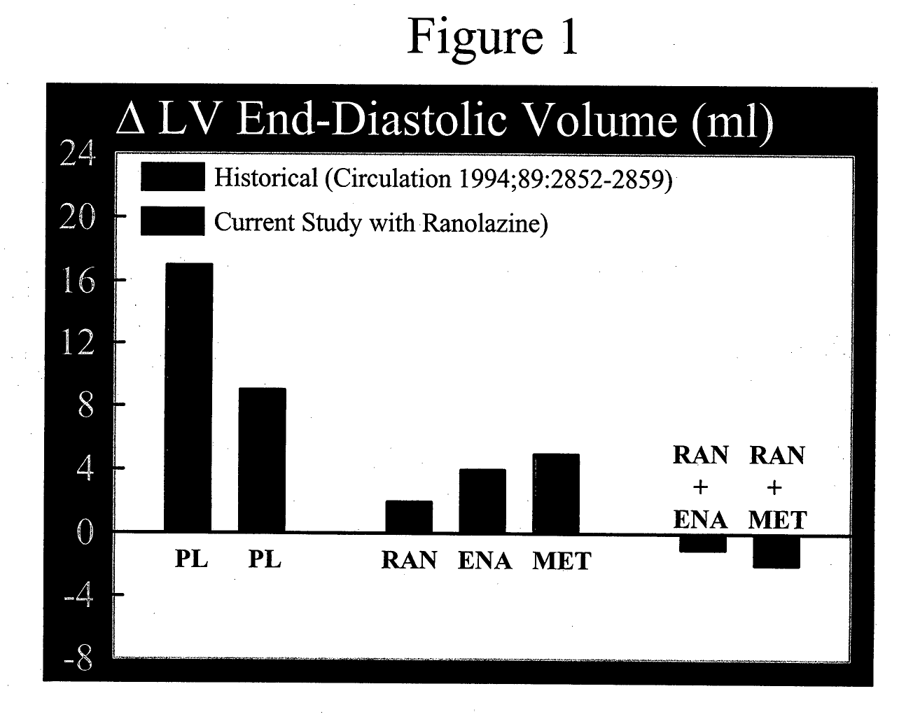

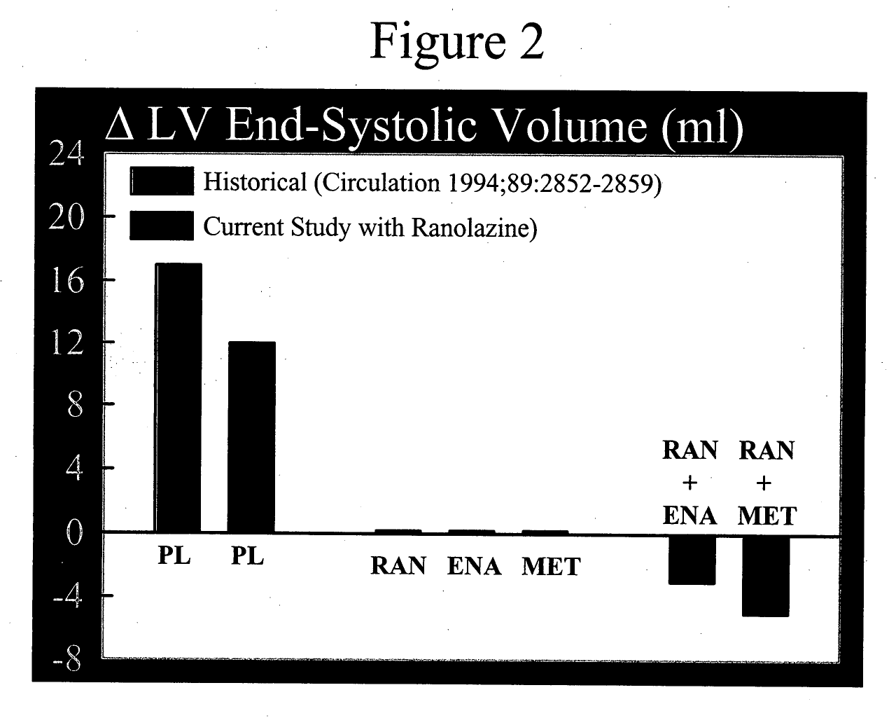

Historical data on the effects of the ACE inhibitor enalapril and the beta-blocker metoprolol on LV reverse remodeling was compared to the data obtained in Example 1 for ranolazine alone, ranolazine and enalapril, and ranolazine and metoprolol tartrate. The enalapril and metoprolol data was taken from Sabbah et al. (1994) Circ. 89:2852-2859. Comparative results are presented graphically in FIGS. 1 and 2.

FIG. 1 illustrates how while neither ranolazine, enalapril, nor metoprolol were independently able to reduce LV end-diastolic volume, combined administration of ranolazine and enalapril and combined administration of ranolazine and metoprolol were able to reduce LV end-diastolic volume, i.e., to reverse LV remodeling.

FIG. 2 illustrates how while neither ranolazine, enalapril, nor metoprolol appear to independently reduce LV end-systolic volume, combined administration of ranolazine and enalapril and combined administration of ranolazine and metoprolol were able to reduce LV end-systolic volume, i.e., to reverse LV remodeling.

We claim:

1. A method for reversing unfavorable left ventricle remodeling comprising administration of a therapeutically effective amount of ranolazine and a therapeutically effective amount of at least one co-remodeling agent to a mammal in need thereof.

2. The method of claim 1, wherein the co-remodeling agent comprises an ACE inhibitor, an ARB, or a beta-blocker.

3. The method of claim 2, wherein the co-remodeling agent comprises an ACE inhibitor.

4. The method of claim 3, wherein the ACE inhibitor is selected from the group consisting of benazepril, captopril, cilazapril, enalapril, fosinopril, imidapril, lisinopril, perindopril, quinapril, ramipril, temocapril, and trandolapril.

5. The method of claim 2, wherein the co-remodeling agent comprises an ARB.

6. The method of claim 5, wherein the ARB is selected from the group consisting of candesartan, cilexetil, eprosartan, irbesartan, losartan, olmesartan, medoxomil, telmisartan, valsartan, zolasartin, and tasosartan.

7. The method of claim 2, wherein the co-remodeling agent comprises a beta-blocker.

8. The method of claim 7, wherein the beta-blocker is selected from the group consisting of, acebutolol, atenolol, betaxolol, bisoprolol, carteolol, labetalol, metoprolol, nadolol, oxprenolol, penbutolol, pindolol, propranolol, sotalol, timolol, esmolol, sotalol, carvedilol, medroxalol, bucindolol, levobunolol, metipranolol, celiprolol, and propafenone.

9. The method of claim 2, wherein the left ventricle remodeling is the result of congestive heart failure (CHF) and/or chronic heart failure.

10. The method of claims 1, wherein the ranolazine and the co-remodeling agent are administered as separate dosage forms.

11. The method of claim 1, wherein ranolazine and the co-remodeling agent are administered as a single dosage form.

12. A pharmaceutical formulation comprising a therapeutically effective amount of ranolazine, a therapeutically effective amount at least one co-remodeling agent, and at least one pharmaceutically acceptable carrier.

13. The formulation of claim 12, wherein the co-remodeling agent comprises an ACE inhibitor, an ARB, or a beta-blocker.

14. The formulation of claim 13, wherein the co-remodeling agent comprises an ACE inhibitor.

15. The formulation of claim 14, wherein the ACE inhibitor is selected from the group consisting of benazepril, captopril, cilazapril, enalapril, fosinopril, imidapril, lisinopril, perindopril, quinapril, ramipril, temocapril, and trandolapril.

16. The formulation of claim 13, wherein the co-remodeling agent comprises an ARB.

17. The formulation of claim 16, wherein the ARB is selected from the group consisting of candesartan, cilexetil, eprosartan, irbesartan, losartan, olmesartan, medoxomil, telmisartan, valsartan, zolasartin, and tasosartan.

18. The formulation of claim 13, wherein the co-remodeling agent comprises a beta-blocker.

19. The formulation of claim 18 wherein the beta-blocker is selected from the group consisting of, acebutolol, atenolol, betaxolol, bisoprolol, carteolol, labetalol, metoprolol, nadolol, oxprenolol, penbutolol, pindolol, propranolol, sotalol, timolol, esmolol, sotalol, carvedilol, medroxalol, bucindolol, levobunolol, metipranolol, celiprolol, and propafenone.

20. A method for treating heart failure comprising administration of a therapeutically effective amount of ranolazine and a therapeutically effective amount of at least one co-remodeling agent to a mammal in need thereof.

21. The method of claim 20, wherein the co-remodeling agent comprises an ACE inhibitor, an ARB, or a beta-blocker.

22. The method of claim 21, wherein the co-remodeling agent comprises an ACE inhibitor.

23. The method of claim 22, wherein the ACE inhibitor is selected from the group consisting of benazepril, captopril, cilazapril, enalapril, fosinopril, imidapril, lisinopril, perindopril, quinapril, ramipril, temocapril, and trandolapril.

24. The method of claim 21, wherein the co-remodeling agent comprises an ARB.

25. The method of claim 24, wherein the ARB is selected from the group consisting of candesartan, cilexetil, eprosartan, irbesartan, losartan, olmesartan, medoxomil, telmisartan, valsartan, zolasartin, and tasosartan.

26. The method of claim 21, wherein the co-remodeling agent comprises a beta-blocker.

27. The method of claim 26, wherein the beta-blocker is selected from the group consisting of, acebutolol, atenolol, betaxolol, bisoprolol, carteolol, labetalol, metoprolol, nadolol, oxprenolol, penbutolol, pindolol, propranolol, sotalol, timolol, esmolol, sotalol, carvedilol, medroxalol, bucindolol, levobunolol, metipranolol, celiprolol, and propafenone.

28. The method of claim 21, wherein the heart failure is congestive heart failure (CHF) or chronic heart failure.

29. The method of claims 20, wherein the ranolazine and the co-remodeling agent are administered as separate dosage forms.

30. The method of claim 20, wherein ranolazine and the co-remodeling agent are administered as a single dosage form.
